(19) United States
(12) Patent Application Publication
(10) Pub. No.: US 2017/0333151 A1
(43) Pub. Date: Nov. 23, 2017

(54) WIDE ANGLE ILLUMINATION SYSTEM AND METHOD

(71) Applicant: H.S. International Corp., Concord, CA (US)

(72) Inventor: Madan Maholtra, Concord, CA (US)

(21) Appl. No.: 15/161,159

(22) Filed: May 20, 2016

(51) Int. Cl. A61B 90/30; A61F 9/007; F21V 8/00

(52) U.S. Cl. CPC A61B 90/30; G02B 6/0008; A61F 9/00736; A61B 2090/306

(57) ABSTRACT

A wide angle illumination system and method. The wide angle illumination system is efficient in facilitating wide angle illumination of interior surfaces during vitreoretinal surgery. An optical fiber terminates in a convex semi-spherical end. A light source transmits a light beam through the optical fiber toward the convex semi-spherical end. An optical element has a flat, straight or planar end that is opposite to and adjoins a convex semi-spherical end which is adjacent to and which faces the convex semi-spherical end of the optical fiber. The light source transmits a light beam through the optical fiber convex semi-spherical end to the semi-spherical end of the optical element after which the convex semi-spherical end of the optical element transmits and diverges the light beam through the flat planar end into the interior of a surgical surface.

### BACKGROUND

The present disclosure relates generally to vitrectomy probes and surgical instruments and more specifically to a vitrectomy probe and a surgical instrument that provides wide angle illumination during a vitreoretinal surgical operation.

Around the world, roughly 250 million people may have some kind of vision impairment that requires removal of vitreous humor from the eye. Vitreous humor also herein referred to as vitreous is a complex and fibrous gel-like substance that fills about 80 percent of the eye and helps to maintain the eye's round shape.

Vitreous removal is accomplished via vitrectomy, a surgical procedure for the eye that involves the placement of ports in the eye through which various instruments can be passed. For example, an illumination system may be passed through one of the ports to illuminate the interior of the eye during a vitreoretinal operation in which vitreous is cut and removed from the eye. As is then apparent, given the importance of the human eye, the procedure must be performed optimally with instruments that facilitate vitrectomy and minimize trauma that can arise during this surgical procedure.

It is within the aforementioned context that a need for the present disclosure has arisen. Thus, there is a need to address one or more of the disadvantages of conventional systems and methods, and the present disclosure meets this need.

### BRIEF SUMMARY

Various aspects of a wide angle illumination system and method can be found in exemplary embodiments of the present disclosure. In one aspect, the wide angle illumination system and method is efficient in facilitating wide angle illumination of interior surfaces of the eye during vitreoretinal surgery.

In one embodiment, among other components, the system includes a preferably lengthy optical fiber that has a first end and a second oppositely disposed end that terminates in a convex semi-spherical end. A light source transmits a light beam through the optical fiber toward the convex semi-spherical end.

The system might also include an optical element with a flat, straight, planar end that is opposite to and adjoins a convex semi-spherical end. In one embodiment, the convex semi-spherical end of the optical element and the convex semi-spherical end of the optical fiber have substantially similar dimensions and shape and are adjacent to and face each other. In this manner, a light beam is transmitted from the convex semi-spherical end of the optical fiber to the convex semi-spherical end of the optical element after which the convex semi-spherical end of the optical element transmits and refracts the light beam through the flat planar end.

In an embodiment, the wide angle illuminator system includes a cannula that houses the optical element and the optical fiber end with a convex semi-spherical end. In another embodiment, the wide angle illuminator system may include a cannula for 20 G, 23 G, 25 G or 27 G. In yet another embodiment, the wide angle illuminator system is such that angles of incidence at which the light rays are received at a surface of the semi-spherical convex end of the optical fiber are greater than angles of incidence at which light rays for a flat surface convex end are received. In another embodiment of the wide angle illuminator system, light rays refracted from the semi-spherical convex end of the optical fiber are at higher angles of refraction than light rays that are reflected from a flat surface.

In another embodiment, a method provides an optical fiber of elongated length. The optical fiber includes a proximal end, and a distal end that terminates in a convex semi-spherical end. A light source is optically coupled to the proximal end of the optical fiber and the light source transmits a light beam through the optical fiber toward the convex semi-spherical end. The method provides an optical element with a planar end that is oppositely disposed to a convex semi-spherical end. The convex semi-spherical ends are dimensioned and shaped to be substantially similar. The convex semi-spherical ends are adjacent and face each other. The convex semi-spherical ends are facing each other so that the light beam is transmitted from the convex semi-spherical end of the optical fiber to the convex semi-spherical end of the optical element. The convex semi-spherical end of the optical element then transmits and diverges the light beam through the planar end.

Further yet, in another embodiment, an apparatus comprises an optical fiber of elongate length wherein the optical fiber includes a proximal end and an opposite distal end that terminates in a convex semi-spherical or conical end; a light source optically coupled to the proximal end of the optical fiber wherein the light source transmits a light beam through the optical fiber toward the convex semi-spherical or conical end; an optical element with a planer end that is oppositely disposed to a convex semi-spherical or conical end wherein the convex semi-spherical or conical end of the optical element and the convex semi-spherical or conical end of the optical fiber are dimensioned and shaped to be substantially similar and wherein the convex semi-spherical or conical end of the optical element and the convex semi-spherical or conical end of the optical fiber are adjacent and face each other wherein the convex semi-spherical or conical ends are facing each other so that a light beam is transmitted from the convex semi-spherical or conical end of the optical fiber to the convex semi-spherical or conical end of the optical element, the convex semi-spherical or conical end of the optical element transmitting and diverging the light beam through the flat exterior surface.

A further understanding of the nature and advantages of the present disclosure herein may be realized by reference to the remaining portions of the specification and the attached drawings. Further features and advantages of the present disclosure, as well as the structure and operation of various embodiments of the present disclosure, are described in detail below with respect to the accompanying drawings. In the drawings, the same reference numbers indicate identical or functionally similar elements.

### BRIEF DESCRIPTION OF THE DRAWINGS

FIG. 1 illustrates a front plan view of a human eye during vitrectomy surgery in accordance with an embodiment of the present disclosure.

FIG. 2 is a cross-sectional view of the human eye of FIG. 1.

FIG. 3 illustrates a wide angle illumination system according to an exemplary embodiment of the present specification.

FIG. 4 illustrates the interior of the cannula needle of FIG. 3 in accordance with an embodiment of this specification.

FIG. 5 illustrates the transmission of input light L′ through the interior of the cannula needle of FIG. 3 in accordance with an embodiment of this specification.

FIG. 6 illustrates the interior of cannula needle of FIG. 3 in accordance with another exemplary embodiment of this specification.

FIG. 7 illustrates an optical fiber pulling system in accordance with an exemplary embodiment of the present specification.

FIG. 8 illustrates an optical fiber with a semi-spherical convex end 802.

FIG. 9 illustrates an optical fiber with a conical end.

### DETAILED DESCRIPTION

Reference will now be made in detail to the embodiments of the disclosure, examples of which are illustrated in the accompanying drawings. While the disclosure will be described in conjunction with the one embodiment, it will be understood that they are not intended to limit the disclosure to these embodiments. On the contrary, the disclosure is intended to cover alternatives, modifications and equivalents, which may be included within the spirit and scope of the disclosure as defined by the appended claims. Furthermore, in the following detailed description of the present disclosure, numerous specific details are set forth to provide a thorough understanding of the present disclosure. However, it will be obvious to one of ordinary skill in the art that the present disclosure may be practiced without these specific details. In other instances, well-known methods, procedures, components, and circuits have not been described in detail as to not unnecessarily obscure aspects of the present disclosure.

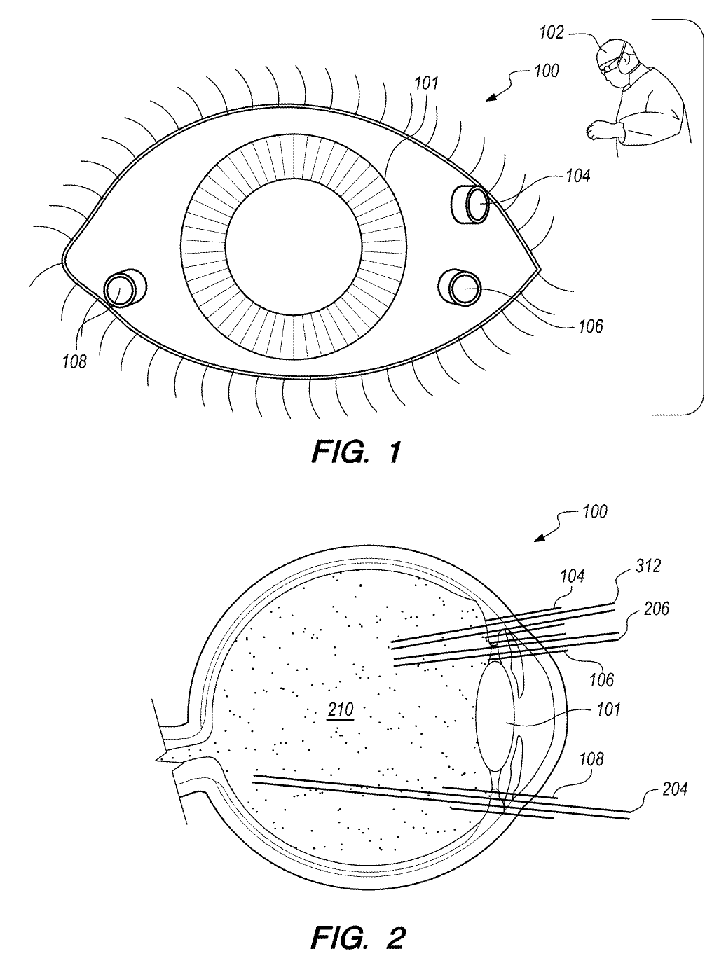

FIG. 1 illustrates a front plan view of human eye 100 during vitrectomy surgery in accordance with an embodiment of the present disclosure.

In FIG. 1, a user or eye surgeon 102 may perform vitrectomy on human eye 100 to rectify vision impairment such as that associated with retinal detachment (for example). This surgical procedure might specifically be performed to remove vitreous humor 210 (see FIG. 2) from human eye 100.

As shown in FIG. 1, eye surgeon 102 begins by inserting a number of ports 104, 106 and 108 adjacent to iris 101. Specifically, the inserted ports are light port 104, saline port 106 and vitrectomy cutter port 108. Here, each port is an entryway for inserting a surgical instrument into human eye 100 as further illustrated with reference to FIG. 2.

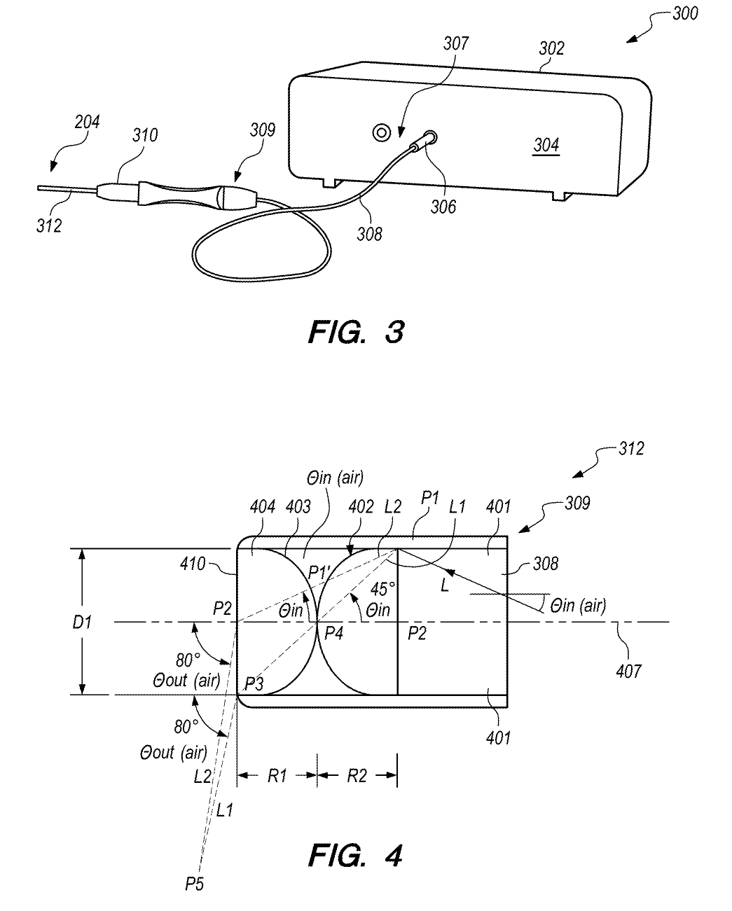

FIG. 2 is a cross-sectional view of human eye 100 illustrating surgery instruments inserted into light port 104, saline port 106 and vitrectomy cutter port 108 of FIG. 1. Here, eye surgeon 102 (of FIG. 1) passes a cannula needle 312 (of wide angle illumination system of FIG. 3) through light port 104 into the interior of human eye 100 as shown. Wide angle illumination system 300 can then be employed to illuminate the interior of human eye 100 and maintain visibility as vitrectomy is performed. Eye surgeon 102 has the flexibility to move and redirect the light probe to the various areas of the eye interior as needed for illumination.

After insertion of cannula needle 312, a saline tube 206 is then passed through saline port 106, the saline tube 206 permitting introduction of saline (or other comparable liquid or gaseous matter) into the eye, thus maintaining the eye's roundness as vitreous humor 210 is removed from human eye 100.

A vitrectomy cutter port 108 is also inserted into human eye 100. As implied by its name, vitrectomy cutter port 108 enables eye surgeon 102 to pass a vitrectomy cutter 204 through vitrectomy cutter port 108 to cut and aspirate vitreous humor 210 from human eye 100.

FIG. 3 illustrates wide angle illumination system 300 according to an exemplary embodiment of the present specification.

In FIG. 3, eye surgeon 102 (FIG. 1) may employ wide angle illumination system 300 to direct an illuminative light beam into the interior of the human eye during any number of intraocular or ophthalmic surgical procedures. Such procedures may include vitrectomy for retinal surgery macular hole, diabetic retinopathy, retinal detachment, uveitis, and age-related macular degeneration for example. Although not shown, one of ordinary skill in the art will realize that embodiments of the present specification can be used for other surgical procedures other than ophthalmic surgical applications.

In FIG. 3, among other components, wide angle illumination system 300 comprises light source 302 optically connected to optical fiber 308 via optical coupling 306. Optical coupling 306 may be a connector that facilitates a secure connection and minimizes loss of light rays traveling from light source 302 to optical fiber 308.

Here, light source 302 can generate a light beam (diffusive) that is then transmitted through optical fiber 308 to illuminate an interior surgical surface. Light source 302 may include illuminative sources such as halogen, LED (Light Emitting Diode), metal halide, mercury vapor and other like sources for performing vitreoretinal surgery as known to those skilled in the art.

As another example, light source 302 may also be based on a xenon source. Light source 302 may also include a combination of different light sources or multiple light sources. For example, multiple LEDs can be blended to provide visible spectral outputs.

As shown, light source 302 may include filter 304 that might be used to eliminate wavelengths (typically shorter wavelengths 420-435 nm) that do not provide illumination but might be phototoxic. Light source 302, in one embodiment, may provide a 40 lumen output to provide robust illumination.

In FIG. 3, optical fiber 308 of wide angle illumination system 300 can be any fiber optic cable known to those skilled in the art but preferably can be 19, 20, 25, or 27 gauge. However, the lower the gauge, the more powerful light source 302 must be. Optical fiber 308 has an NA (numerical aperture) of about 0.05, which is suitable for most ophthalmic applications. Here, NA is the angle of acceptance of entrance of the light beam from light source 302 into optical fiber 308.

Referring to FIG. 3, optical fiber 308 has proximal end 307 optically coupled to light source 302 and an oppositely disposed distal end 309 connected to handle or housing 310. Handle 310 is itself connected to cannula needle 312. In one embodiment, distal end 309 of optical fiber 308 extends through handle 310 and terminates at the tip of cannula needle 312. As will be discussed with reference to FIG. 4, optical fiber 308 terminates at a semi-spherical convex tip or end.

Optical fiber 308 further has an elongated length. However, any suitable length may be employed, so long as such length permits easy manipulation of cannula needle 312 by eye surgeon 102 during a vitreoretinal operation.

As noted, wide angle illumination system 300 includes handle 310 and cannula needle 312. As indicated by its name, handle 310 allows eye surgeon 102 to grip and manipulate cannula needle 312 during a surgical operation; handle 310 further provides a housing for delivering the light beam from the distal end of optical fiber 308 to the tip of cannula needle 312. As further discussed with reference to FIG. 4, specifically, cannula needle 312 houses both the distal end 309 of optical fiber 308 and optical element 404 (FIG. 4) that accepts light rays from optical fiber 308 and emits the light rays into the interior of the eye during surgery.

FIG. 4 illustrates the interior of cannula needle 312 of FIG. 3 in accordance with an embodiment of this specification.

In FIG. 4, cannula needle 312 comprises optical fiber 308 (distal end 309 of optical fiber 308) and optical element 404—both of which are disposed in an adjacent relationship with each other. As can be seen, optical fiber 308 terminates in a semi-spherical or fiber convex end 402 that faces optical element 404. Optical element 404 itself is composed of two adjoining ends. The first is a planar end 410 having a plane that is substantially perpendicular to optical axis 407 (of optical element 404 and optical fiber 308). The second end is a semi-spherical optical element convex end 403 oppositely disposed from planar end 410.

Optical element 404 may be sapphire or any other comparable material consistent with the spirit and scope of the present specification. Preferably, the diameter D1 of both the optical element 404 and optical fiber 308 may be 0.25 mm to 0.75 mm. The radius R1 of optical element 404 and the radius R2 of optical fiber 308 may range from 0.127 mm to 0.381 mm for 27 G to 20 gauge fiber. One of ordinary skill in the art will realize that the stated dimensions may depend upon the gauge employed. Non-limiting examples of the gauges include 20 G, 23 G, 25 G. Other gauges such as 27 G may be employed, for example.

As shown in FIG. 4, optical element convex end 403 and fiber convex end 402 are adjacent and face each other. They are also configured to have substantially similar dimensions in addition to being configured to have substantially similar configuration or shape. In this manner, the convex ends support each other to receive and refract input light rays that produce a maximum angular spread.

In the embodiment shown, both ends are arranged to touch each other. Thus, a light beam traveling from light source 302 (FIG. 3) through optical fiber 308 is transmitted from fiber convex end 402 to optical element convex end 403 where the light beam is received; in turn optical element convex end 403 transmits and refracts the light beam through planar end 410 at both an illumination and an angular spread that are unlike conventional systems.

Specifically, unlike conventional systems, an advantage of an embodiment of the present specification is that not only is the intensity of illumination relatively high (see e.g. Table 1 and Table 2 below), the angular spread of the emanating light rays is wide. Thus, the light beam emitted by planar end 410 has a maximum half angle of 80 degrees from optical axis 407. In total, the angular spread obtained by an embodiment of the present specification is 80+80 degrees for a total of 160 degrees.

In operation, input light L from air (with a maximum half angle θᵢₙ₍ₐᵢᵣ₎ within the acceptance angle range of optical fiber 308) is admitted and reflected at point P1. Point P1 is the upper reflective surface of optical fiber 308 over which cladding 401 is disposed. At point P1, input light L may be reflected as single or multiple light rays L1 and L2. One of skill in the art will realize that the reflection of L1 and L2 is for exemplary purposes.

Point P1 also marks the point where the convex shape of fiber convex end 402 begins. Unlike prior art light pipe systems, optical fiber 308 includes this semi-spherical convex surface of fiber convex end 402 that is complementary with the semi-spherical convex surface of optical element convex end 403.

Here, the upper reflective surface of optical fiber 308 reflects light L2 from point P1 through P1′ to point P2. Point P2, which lies on optical axis 407, is also positioned at one half the diameter D1 of optical element 404 on planar end 410. From point P2, L2 is refracted to point P5 into air at a maximum half angle of θₒᵤₜ₍ₐᵢᵣ₎ here, approximately 80 degrees as shown. Thus, a novel wide angular displacement obtained by an embodiment of the present specification is 80+80 degrees for a total of 160 degrees.

Similarly, the upper reflective surface of optical fiber 308 reflects light L1 through point P4 on optical axis 407 to point P5 in air. Point P4 lies in the intersection of the midpoints of both convex exterior surfaces where both surfaces touch each other. At this point P4, light L1 is transmitted straight through without refraction as light L1 travels from optical fiber 308 to optical element 404 without traveling through air (which has a lower refractive index optical fiber 308 or optical element 404).

Further, at point P4, light L1 is incident at an angle θᵢₙ of 45 degrees so that upon arriving at point P3, light L1 (like light L2) is refracted to point P5 in air at a maximum half angle of θₒᵤₜ₍ₐᵢᵣ₎ here, approximately 80 degrees as shown.

Point P3 lies on planar end 410 where optical element 404 transitions to air from sapphire—one medium of optical element 404. Thus, at point P3, L1 is refracted into air at a maximum half angle of θₒᵤₜ₍ₐᵢᵣ₎ to point P5.

As can be seen, an advantage is derived by having fiber convex end 402 with its semi-spherical surface facing that of optical element convex end 403. Without fiber convex end 402, much of the light received and emitted through optical fiber 308 will simply pass straight through optical element 404 forming a straight light pipe as is well known in the art.

In one embodiment, by providing this fiber convex end 402 that is complementary with optical element convex end 403, wide angle illumination system 300 increases angular spread in accordance with Snell's law. Briefly, Snell's law states that the ratio of the sines of the angles of incidence and refraction is equivalent to the ratio of phase in the two media or equivalent to the reciprocal of the ratio of the indices of refraction. The complementary nature of fiber convex end 402 and optical element convex end 403 will now be described.

Fiber Convex End 402 Receives Input Light Ray in Optic Fiber Media (e.g. Glass) and Refracts Input Ray into Air: In accordance with Snell's law, when light rays—e.g., L1 and L2 of FIG. 4—are received on the surface of fiber convex end 402 at angles of incidence, the light rays are refracted at angles of refraction greater than the angles of incidence since the light rays are travelling from glass (the optical fiber medium) to air. Here, in particular, the angles of incidence at which the light rays are received at the surface of fiber convex end 402 are typically greater (compared to a flat surface) because the surface of fiber convex end 402 is semi-spherical.

Therefore, since the light rays incident on this semi-spherical surface of fiber convex end 402 are at increased angles of incidence, the resulting refracted light rays also have increased angles of refraction into air. In other words, the light rays refracted from fiber convex end 402 are at higher angles of refraction than light rays that are reflected from a flat conventional surface.

Optical Element Convex End 403 Receives Light Ray from Air and Refracts Light Ray through Optical Element Media (e.g. Sapphire): After refraction into air by fiber convex end 402, the light rays are then incident on optical element convex end 403 which refracts the light rays through it.

In accordance with embodiments of the present disclosure, when the light rays (refracted from fiber convex end 402) are incident on optical element convex end 403, the rays are: 1) at locations that are different from where they would have been had the light rays been refracted from a flat conventional surface, and 2) the rays are at increased angles of incidence at optical element convex end 403 due to the increased angles of refraction from the fiber convex end 402.

The increased angles of incidence caused by the novel semi-spherical surface of fiber convex end 402 minimizes the amounts by which the angles of refraction are reduced when the rays are refracted through optical element convex end 403. Specifically, the angles of refraction of the light rays through optical element convex end 403 are less than the angles of incidence since the light rays are travelling from air to sapphire (for example). Therefore, the increased angles of incidence caused by the novel semi-spherical surface of fiber convex end 402 minimize the amounts by which the angles of refraction are reduced.

In accordance with embodiments of the present disclosure, another advantage is that the semi-spherical surface of optical element convex end 403 also complements the semi-spherical surface of fiber convex end 402 by increasing the angles of incidence of the light rays on optical element convex end 403 so as to minimize the amounts by which the angles of refraction are reduced. In this manner, the refracted light through the optical element convex end 403 can diverge (with maximum angles of refraction) and become incident on the planar end 410 at higher angles of incidence.

Planar End 410 (of Optical element Convex End 403) Receives Input Ray from Optical Element Media (e.g. Sapphire) and Diverges into Air:

The light rays that are refracted and become incident on planar end 410 are refracted into air at angles of refraction greater than the angles of incidence since the light rays are travelling from sapphire (the optical element medium) to air. Here, in particular, the angles of incidence at which the light rays are received at the surface of planar end 410 are higher because the complementary surface of optical element convex end 403 has minimized its refraction (through the optical element media—sapphire) thus maximizing the incidence angles at the planar end 410. As a result, the light rays that are refracted into air at planar end 410 are configured to have a maximum angular spread.

Thus, the present specification offers the advantage of a semi-spherical convex optical fiber end complementary with a semi-spherical optical element that cannot only provide wide angular spreads, the present disclosure also significantly improves illumination of the wide angular illumination system relative to conventional systems.

Table 1 shows critical results indicating light output illumination of applicant's 20 G wide angle illumination system over a conventional 20 G wide angle probe.

TABLE 1

| 20G Wide Angle Probe | Light Output (Lux) |
|---|---|
| Conventional | 364 |
| HSI—Applicant's Wide Angle | 578 |
| Illumination System | |

Table 1 shows the light output in Lux of a conventional 20 G wide angle probe relative to applicant's wide angle illumination system of the present disclosure. As can be seen, the light output illumination of applicant's wide angle illumination system shows an unexpected result of 578 Lux which is much higher than that of the conventional wide angle probe which was 364 Lux.

Table 2 shows critical results indicating light output illumination of applicant's 23 G wide angle illumination system over a conventional 23 G wide angle probe.

TABLE 2

| 23G Wide Angle Probe | Light Output |
|---|---|
| Conventional | 144 |
| HSI—Applicant's Wide Angle | 287 |
| Illumination System | |

As can also be seen, the light output or illumination of applicant's wide angle illumination system is an unexpected 287 Lux much higher than that of the conventional wide angle probe which was is 144 Lux. Thus, the present specification is advantageous and provides not only a wide angular spread, but the present disclosure also significantly improves illumination of the wide angular illumination system over conventional systems.

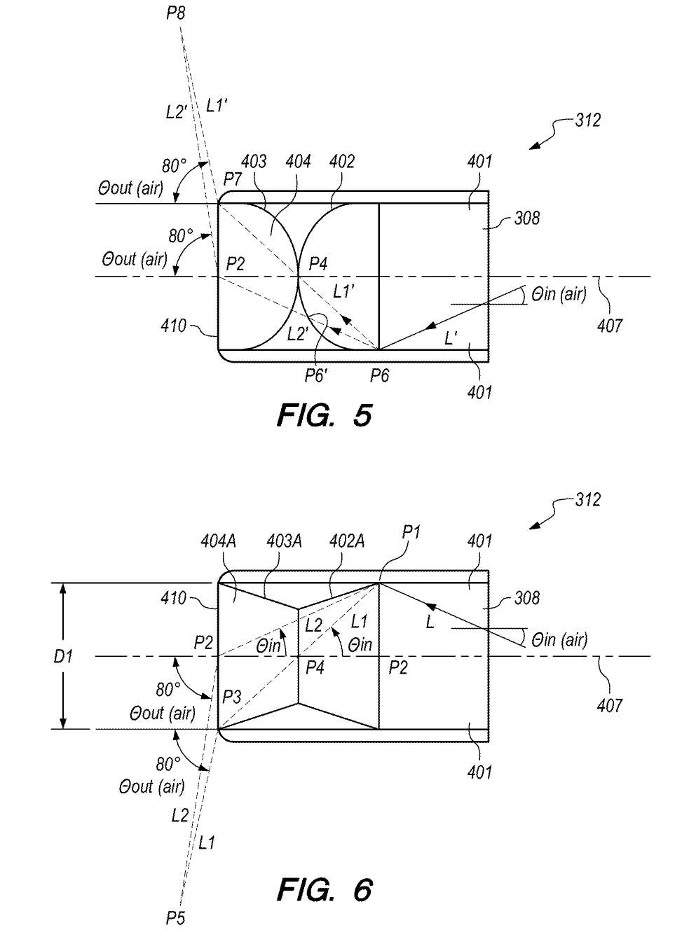

FIG. 5 illustrates the transmission of input light L′ through the interior of cannula needle 312 of FIG. 3 in accordance with an embodiment of this specification.

In FIG. 5, as in FIG. 4, input light L′ that has a maximum half angle angle θᵢₙ₍ₐᵢᵣ₎ is received and reflected at point P6. Point P6 is on a lower reflective surface of optical fiber 308 on which cladding 401 is disposed.

In particular, P6 is located at the point where fiber convex end 402 begins to curve to provide reflection. From point P6, the lower reflective surface reflects two light rays L1′ and L2′. L1′ is transmitted through point P4. Point P4 is on optical axis 407 where the adjacent faces of optical element convex end 403 and fiber convex end 402 are in contact. From point P4, L1′ is transmitted to P7 where L1′ is emitted into air at a maximum half angle θₒᵤₜ₍ₐᵢᵣ₎ of 80 degrees to point P8.

Referring back to point P6, the reflective lower surface transmits L2′ through P6′ to point P2. At point P6′, L2′ may be refracted in air (although this is not illustrated). From point P2, L2′ is emitted into air at a maximum half angle θₒᵤₜ₍ₐᵢᵣ₎ of 80 degrees to point P8.

FIG. 6 illustrates the interior of cannula needle 312 of FIG. 3 in accordance with another exemplary embodiment of this specification.

In FIG. 6, the interior of cannula needle 312 houses an optical element 404A that has a cone tapered end 403A. This cone tapered end 403A is adjacent to and faces optical fiber 308A that has a cone tapered end 402A. As in the embodiment of FIG. 4, input light L from the air is reflected at P1 via light ray L1 and light ray L2 with light ray L2 being emitted at P2 at a maximum half angle of 80 degrees and L1 being emitted into the air at P3 at a maximum half angle of 80 degrees. Otherwise, the embodiment of FIG. 6 functions in a manner that is similar to the embodiment of FIG. 4.

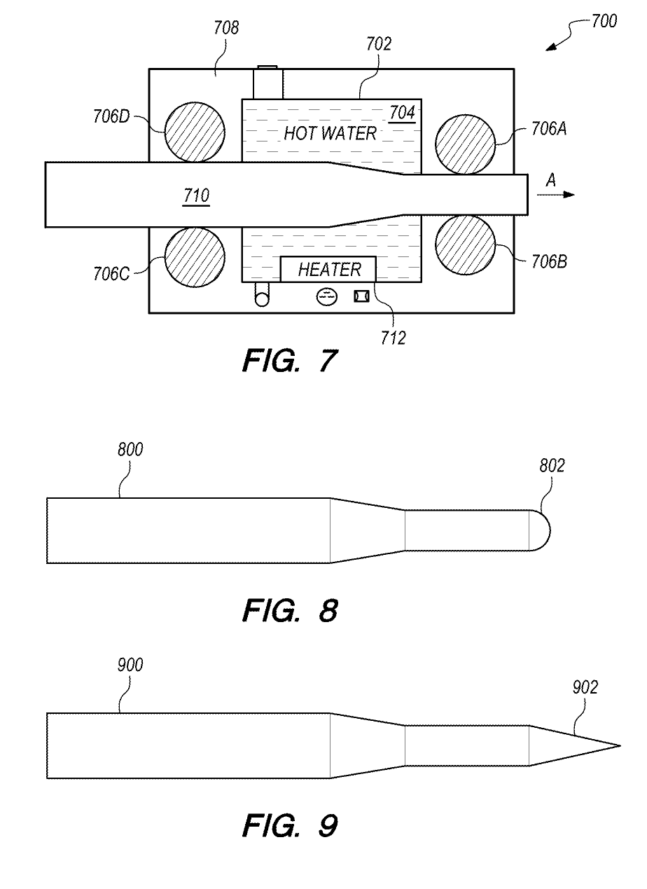

FIG. 7 illustrates an optical fiber pulling system 700 in accordance with an exemplary embodiment of the present specification.

In FIG. 7, optical fiber pulling system 700 may be used to stretch optical fibers and to shape optical fibers for use with embodiments of the present specification. Unlike the prior art, optical fiber pulling system 700 utilizes a hot water chamber 702 to provide wet heat optical fiber pulling to avoid cracking of optical fiber during the pulling process. Hot water chamber 702 includes a heater 712 for heating up the hot water 704 that is contained within the chamber. Optical fiber pulling system 700 further includes a plurality of o-rings 706A, 706B, 706C, and 706D. O-rings 706A and 706B are located at the forefront of the optical fiber pulling system 700. Specifically, the entirety of the system of the hot water chamber 702 and the o-rings 706 are enclosed within an acrylic glass chamber 708. Acrylic glass chamber 708, o-rings 706A and 706B are located at the forefront of acrylic glass chamber 708. O-rings 706C and 706D are located at the back of acrylic glass chamber 708.

In operation, optical fiber 710 that is to be stretched is first positioned between o-rings 706C and 706D. The optical fiber 710 is then attached to a PLC (programmable logic controller) motion controller linear motion slider that pulls optical fiber 710 at a distal end through the o-rings along the direction A while an oppositely disposed proximal end of the optical fiber 710 remains fixed. Optical fiber 710 is pulled for 5 minutes through hot water chamber 702 while hot water chamber 702 is maintained at 100° C. Therafter, optical fiber 710 is pulled through the o-rings 706A and 706B.

FIG. 8 illustrates optical fiber 800 with a semi-spherical convex end 802. FIG. 9 illustrates optical fiber 900 with a conical end 902. Both optical fiber 800 and optical fiber 900 may be produced via the apparatus of FIG. 7.

While the above is a complete description of exemplary specific embodiments of the disclosure, additional embodiments are also possible. Thus, the above description should not be taken as limiting the scope of the specification, which is defined by the appended claims along with their full scope of equivalents.

I claim:

1. A wide angle illuminator system comprising an optical fiber of elongate length wherein the optical fiber includes a proximal end and an opposite distal end that terminate in a convex semi-spherical end;
a light source optically coupled to the proximal end of the optical fiber wherein the light source transmits a light beam through the optical fiber toward the convex semi-spherical end;
an optical element with a planar end that is oppositely disposed to a convex semi-spherical end wherein the convex semi-spherical end of the optical element and the convex semi-spherical end of the optical fiber are dimensioned and shaped to be substantially similar; and
wherein the convex semi-spherical end of the optical element and the convex semi-spherical end of the optical fiber are adjacent and face each other wherein the convex semi-spherical ends are facing each other so that a light beam is transmitted from the convex semi-spherical end of the optical fiber to the convex semi-spherical end of the optical element wherein the convex semi-spherical end of the optical element transmits and diverges the light beam through the planar end.

2. The wide angle illuminator system of claim 1 further comprising a cannula that houses the optical element and distal end of the optical fiber that terminates in a convex semi-spherical end.

3. The wide angle illuminator system of claim 2 wherein the cannula is for a 20 G, 23 G, 25 G or 27 G.

4. The wide angle illuminator system of claim 1 wherein angles of incidence at which the light rays are received at a surface of the semi-spherical convex end of the optical fiber are greater than a flat surface convex end.

5. The wide angle illuminator system of claim 1 wherein light rays refracted from the semi-spherical convex end of the optical fiber are at higher angles of refraction than light rays that are reflected from a flat surface.

6. A method comprising
providing an optical fiber of elongate length wherein the optical fiber includes a proximal end and an opposite distal end that terminates in a convex semi-spherical end;
optically coupling a light source to the proximal end of the optical fiber wherein the light source transmits a light beam through the optical fiber toward the convex semi-spherical end;
providing an optical element with a planar end that is oppositely disposed to a convex semi-spherical end wherein the convex semi-spherical end of the optical element and the convex semi-spherical end of the optical fiber are dimensioned and shaped to be substantially similar; and
wherein the convex semi-spherical end of the optical element and the convex semi-spherical end of the optical fiber are adjacent and face each other wherein the convex semi-spherical ends are facing each other so that a light beam is transmitted from the convex semi-spherical end of the optical fiber to the convex semi-spherical end of the optical element, the convex semi-spherical end of the optical element transmitting and diverging the light beam through the planar end.

7. The method of claim 6 further comprising providing a cannula that houses the optical element and distal end of the optical fiber that terminates in a convex semi-spherical end.

8. The method of claim 7 wherein the cannula is for a 20 G, 23 G, 25 G or 27 G.

9. The method of claim 6 wherein angles of incidence at which the light rays are received at a surface of the semi-spherical convex end of the optical fiber are greater than a flat surface convex end.

10. The method of claim 6 wherein light rays refracted from the semi-spherical convex end of the optical fiber are at higher angles of refraction than light rays that are reflected from a flat surface.

11. An apparatus comprising
an optical fiber of elongate length wherein the optical fiber includes a proximal end and an opposite distal end that terminates in a convex semi-spherical or conical end;
a light source optically coupled to the proximal end of the optical fiber wherein the light source transmits a light beam through the optical fiber toward the convex semi-spherical or conical end;
an optical element with a planar end that is oppositely disposed to a convex semi-spherical or conical end wherein the convex semi-spherical or conical end of the optical element and the convex semi-spherical or conical end of the optical fiber are dimensioned and shaped to be substantially similar; and
wherein the convex semi-spherical or conical end of the optical element and the convex semi-spherical or conical end of the optical fiber are adjacent and face each other wherein the convex semi-spherical or conical ends are facing each other so that a light beam is transmitted from the convex semi-spherical or conical end of the optical fiber to the convex semi-spherical or conical end of the optical element, the convex semi-spherical or conical end of the optical element transmitting and diverging the light beam through the planar end.

12. The apparatus of claim 11 further comprising a cannula that houses the optical element and distal end of the optical fiber that terminates in the convex semi-spherical or conical end.
